(19) United States
(12) Patent Application Publication
(10) Pub. No.: US 2019/0045903 A1
(43) Pub. Date: Feb. 14, 2019

(54) PROTECTIVE FILM FOR APPLICATION TO AN INDIVIDUALS BODY

(71) Applicant: Advanced Nail Systems, Inc., Minden, NV (US)

(72) Inventors: Monica Ladd-Kimbrough, Biloxi, MS (US); Beth Hickey, Arlington, TX (US); Mark H. Jones, Gardnerville, NV (US)

(21) Appl. No.: 16/164,746

(22) Filed: Oct. 18, 2018

Related U.S. Application Data
(63) Continuation-in-part of application No. 15/299,902, filed on Oct. 21, 2016.
(60) Provisional application No. 62/366,451, filed on Jul. 25, 2016.
(60) Provisional application No. 62/359,409, filed on Jul. 7, 2016.
(60) Provisional application No. 62/302,913, filed on Mar. 3, 2016.
(60) Provisional application No. 62/244,358, filed on Oct. 21, 2015.

(51) Int. Cl. A45D 29/00; A61K 9/70

(52) U.S. Cl. CPC A45D 29/00; A61Q 3/02; A61K 9/703

(57) ABSTRACT

A protective film for application to a portion of an individual's body having a top surface and a bottom surface opposite the top surface. An adhesive is disposed on the top surface for securing the protective film to a portion of an individual's body. An active ingredient is disposed on the top surface and mixed with the adhesive and configured to be disposed onto the portion of the individual's body where the protective film is applied. The protective film has a heel patch portion that generally has a U-shape for surrounding a heel of the individual. According to yet another aspect of the disclosure, the active ingredient includes a solution of water and glycerin with the following ingredients contained therein: mandelic acid at 10-20% in weight, salicylic acid at 2% in weight and bromelain at 10-25% in weight.

### CROSS REFERENCE TO RELATED APPLICATIONS

The present disclosure is a continuation-in-part of U.S. application Ser. No. 15/299,902 filed on Oct. 21, 2016, which claims the benefit and priority to each of the following: U.S. Provisional Patent Application Ser. No. 62/244,358 entitled “An Artificial Nail Apparatus Configured to Deliver a Health Additive to a Fingernail”, filed on Oct. 21, 2015; U.S. Provisional Patent Application Ser. No. 62/302,913 entitled “Protective Nail Film”, filed on Mar. 3, 2016; U.S. Provisional Patent Application Ser. No. 62/359,409 entitled “Protective Nail and Skin Film”, filed on Jul. 7, 2016; and U.S. Provisional Patent Application Ser. No. 62/366,451 entitled “Protective Nail and Skin Film”, filed on Jul. 25, 2016, which are each hereby incorporated by reference in their entirely as though fully set forth herein.

### TECHNICAL FIELD OF THE DISCLOSURE

The present disclosure generally relates to a protective film for application to a portion of an individual's body and, in particular, for application to the individual's nail or skin. The present disclosure also generally relates to a protective film having one or more active ingredients incorporated therein for enhancing the condition of the nail or skin by repairing or minimizing the damage caused by, and/or protecting the nail or skin from, external elements or products.

### BACKGROUND OF THE DISCLOSURE

Daily contact with products having artificial ingredients can cause damage to an individual's body over time. This is particularly true when it comes to the application, long time wear, and removal of nail polish on an individual's finger or toe nails.

Nail polishes date back centuries and have been used as a way for people to decorate their nails. These polishes generally consist of a lacquer or gel that can be applied to a user's finger or toe nails. These polishes are known to come in different colors so that an individual can change the color of their nails, such as to coordinate with an outfit or the like. To provide durability from cracking, chipping, etc., these polishes often include additives or other chemicals to make them harder and more durable. Certain health concerns have been attributable to the use of nail polishes, particularly if the use is extensive.

The process of removing nail polish from a user's nails is also known to create potential damage to a user's health as well as to the nails themselves. In particular, damage can be caused to the various layers of the nail. As shown in FIGS. 1A and 1B, a finger nail 10 can include various layers, such as the dorsal (upper layer) 12, intermedial (middle layer) 14, and the ventral (lower layer) 16. Typically, nail polish is removed with nail pads or nail polish removers. Nail polish removers typically consist of an organic solvent, but may also include oils, scents, and/or coloring. Nail polish remover packages are also known. One type of remover package includes individual felt pads soaked in a chemical remover that can be rubbed against the nail to remove the polish. Another type of remover is known to consist of a bottle of liquid remover that can be applied to a cotton ball or cotton pad, which in turn can be applied to the nail to remove the polish. Still another type of remover can consist of a container filled with foam that allows a user to insert a finger into the container to engage the foam and then twist the finger until the polish comes off. Choosing a type of remover is often determined by the user's preference and often the price or quality of the remover. The most common solvent used in these removers is an acetone, which is a powerful and effective chemical that can be harsh on both skin and nails.

Given the known issues with applying and removing nail polish from an individual's nails, including potential damage thereto, other ways of decorating nails have been developed including gel polishes and artificial or acrylic nails.

Gel polishes are very popular as they are known to last a long time on the nail plate and have increased durability and improved performance over conventional nail polishes. To apply gel polishes, the nail plate is first buffed lightly then the gel is applied and cured with an Ultra Violet or LED light. Additionally, these gel polishes continue to cure with any UV light or LED light including the sun outside, which further increases its durability. This makes the gel harder and the bond to the underlying nail plate tighter. Gel polishes are also known to create difficulty with respect to the removal process. The process for removing the gel generally may consist of soaking the nail and attached gel in a solvent which is made up of acetone for a period of time. Another known removal process includes wrapping and securing cotton balls or pads soaked in acetone around the finger and fingernail for a period of time. After approximately thirty minutes of soaking, a metal scraper is generally used to dig, peel, pry, or scrape the gel off. Because the gel has bonded to the nail, 2 to 3 of the seven layers (as shown in FIG. 1) of the nail plate are often subsequently removed, which can lead to severe nail damage and brittleness over time. Still another known removal process involves drilling the gel off the nail, which can also be very damaging to the user's nail.

Similarly, artificial fingernails are known in the art for overlying natural fingernails to provide for a desired aesthetic affect. Conventional artificial fingernails are typically made of an opaque and hard plastic. These nails are generally attached to the natural fingernails with an adhesive and can be detached from the natural fingernails by later removing the adhesive. After the artificial nails are applied, they are typically coated with a nail polish and/or a finishing material. Alternatively, after the artificial nails are applied, acrylic may be applied over the entire nail with nail polish or finishing material being applied afterwards. The nail polish can be used to make the artificial nails look like the natural nails, although it can also be used to provide other attractive and unique artificial nail appearances. The processes for the removal of artificial or acrylic nail are generally the same as the process for the removal of gel polish and thus suffer the same disadvantages.

Another issue with conventional artificial or acrylic nails is that they may inhibit the healthy development of the underlying natural fingernail and may even allow for the growth of bacteria or other harmful organisms between the natural fingernail and the artificial or acrylic nail.

Accordingly, there remains a need for an improved artificial or acrylic nail. It would thus be desirable to provide a system or method for decorating nails that improves on prior decorative nail processes as well as enhances the natural nail, protects the natural nail from lacquer polishes, gel polishes, and acrylics, protects the natural nail during removal of the same, and acts as a releasing mechanism for the same as well as provides an improved artificial nail.

In addition to nail polishes and similar treatment doing damage to the nail, the same may do damage to the surrounding skin. Further, other products, chemicals, external elements such as friction caused by clothing or shoes, or environmental elements may also cause damage to the skin. These can all cause the skin to crack or the formation of calluses, blisters, or the like. For instance, an individual may develop a callus on their foot because of the shoes they wear.

As such, it would thus be desirable to provide a system or method that not only protects and repairs the nail from decorative nail processes, but can also repair the surrounding skin or other portion of the user's skin that have cracks, calluses, blisters, and the like.

### SUMMARY OF THE DISCLOSURE

It is therefore an aspect of the present disclosure to provide a protective film for application to an individual's finger and toe nails.

It is another aspect of the present disclosure to provide a protective film for application to an individual's body such as a heel, toe or ball of the individual's foot.

It is still another aspect of the present disclosure to provide a protective film that easily confirms to different locations of a user's body to help maintain engagement therebetween.

It is a further aspect of the present disclosure to provide a protective film for application to an individual's toe or finger nails that includes an active ingredient that promotes healing thereof and prevents damage thereto.

It is a related aspect of the present disclosure to provide a protective film for application to an individual's body that includes an active ingredient that is configured to eliminate calluses from the individual's skin.

According to these and the other aspects of the disclosure, a protective film for application to a portion of an individual's body is provided. The protective film includes a heel patch portion that has a first surface and a second surface opposite the top surface. An adhesive is disposed on the first surface for securing the protective film to the portion of the individual's body. An active ingredient is disposed on the first surface and mixed with the adhesive and configured to be disposed onto the portion of the individual's body at the location where the protective film is applied. The heel patch portion generally has a U-shape for surrounding a heel of the individual.

According to yet another aspect of the disclosure, a protective film for application to a portion of an individual's body is provided. The protective film includes a first surface and a second surface opposite the first surface. An active ingredient is disposed on the first surface and configured to be disposed onto the portion of the individual's body at the location where the protective film is applied. The active ingredient includes a solution of water and glycerin with the following ingredients contained therein: mandelic acid at 10-20% in weight, salicylic acid at 2% in weight and bromelain at 10-25% in weight, potassium hydroxide and other actives.

According to yet another aspect of the disclosure, a protective device for application to a portion of an individual's body is provided. The protective device includes a runner including an elongated central hub portion and a plurality of protective film portions extending from the central hub. Each of the protective film portions includes a first edge at the central hub portion and a second edge opposite the hub portion. Each of the protective film portions includes a first side and a second side opposite the first side. An active ingredient is disposed on the second side of each of the protective film portions and is configured to be disposed onto the portion of the individual's body where the protective film is applied. An adhesive is disposed on the second side of each of the protective film portions for securing the protective film portion to the individual's body. A backing membrane overlies the adhesive and the active ingredient of each of the protective film portions. Each of the protective film portions includes a first handle at the first edge and a second handle at the second edge for allowing the individual to remove the backing membrane from the bottom side of the protective film portion.

### BRIEF DESCRIPTION OF THE DRAWINGS

Other aspects of the present disclosure will be readily appreciated, as the same becomes better understood by reference to the following detailed description when considered in connection with the accompanying drawings wherein:

FIG. 1A is a schematic illustration of the anatomy of a nail of an individual;

FIG. 1B is an enlarged view of the area in the circle labeled 1B in FIG. 1A;

FIG. 2 is a top view of an exemplary protective film for application to an individual's body in accordance with an aspect of the present disclosure;

FIG. 3 is a cross-sectional view of an exemplary protective film in accordance with an aspect of the present disclosure;

FIG. 4 is an illustration of examples of various shapes of a protective film for application to an individual's body in accordance with an aspect of the present disclosure;

FIG. 5 is an illustration of an example of the application of a protective film to an individual's nail in accordance with an aspect of the present disclosure;

FIG. 6 is an illustration of an example of a protective film applied to an individual's foot in accordance with an aspect of the present disclosure;

FIG. 7 is a flowchart of a method for applying a protective film to an individual's body in accordance with an aspect of the present disclosure;

FIG. 7A is a substep of the method for applying a protective film of FIG. 7;

FIG. 7B is another substep of the method for applying a protective film of FIG. 7;

FIG. 8 is an exploded perspective view of a carrying sheet with a protective film for application on a fingernail in accordance with an aspect of the present disclosure;

FIG. 9 is a bottom view of a carrying sheet with a protective film for application to an individual's fingernail or toe nail in accordance with an aspect of the present disclosure;

FIG. 10 is a top view of a carrying sheet with a protective film for application to an individual's body for treating a callus in accordance with an aspect of the present disclosure; and

FIG. 11 is a schematic illustration of a carrying sheet with a plurality of sets of protective films for application to an individual's nails.

FIG. 12 is a magnified view of a protective film portion of FIG. 11 with a portion of a backing membrane of the protective film portion cut away.

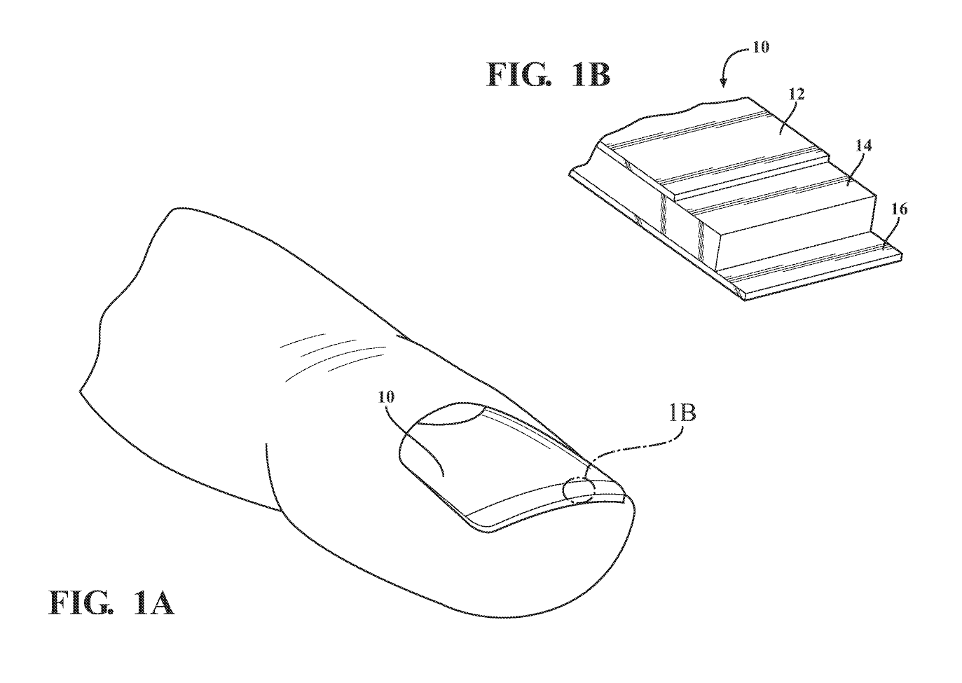

### DETAILED DESCRIPTION OF THE PRESENT DISCLOSURE

Detailed aspects of the present disclosure are provided herein; however, it is to be understood that the disclosed aspects are merely exemplary and may be embodied in various and alternative forms. It is not intended that these aspects illustrate and describe all possible forms of the disclosure. Rather, the words used in the specification are words of description rather than limitation, and it is understood that various changes may be made without departing from the spirit and scope of the disclosure. As those of ordinary skill in the art will understand, various features of the present disclosure as illustrated and described with reference to any of the Figures may be combined with features illustrated in one or more other Figures to produce examples of the present disclosure that are not explicitly illustrated or described. The combinations of features illustrated provide representative examples for typical applications. However, various combinations and modifications of the features consistent with the teachings of the present disclosure may be desired for particular applications or implementations. Additionally, the features and various implementing embodiments may be combined to form further examples of the disclosure.

A protective film 100 for application to a portion of an individual's body, a method of applying a protective film 100 to a portion of an individual's body, and a sheet 130 with a protective film 100 for application to a portion of individual's body are provided. According to an aspect, the protective film 100 and sheet 130 with the protective film 100 are designed to be applied to an individual's nail 116 or a portion of the individual's skin 118. The protective film 100 may also be designed to act as a protective barrier from damage caused by the application or removal of decorative treatment on the individual's nail 116, or one or more external elements against the portion of the individual's skin 118 such as friction generated by the individual clothes or shoes. According to an aspect, the protective film 100 may include an active ingredient 114 which may be one or more health additives for enhancing, improving, protecting or repairing the condition of the individual's nail 116 or skin 118. The protective film 100 may further act as a releasing mechanism for easy removal of the decorative treatments from the nail 116. Accordingly, use of the protective film 100, the method of applying the protective film 100, or apparatus 130 with the protective film 100 provides health benefits to the individual as well as minimizes the negative effects, discussed above, caused by decorative treatments or external elements.

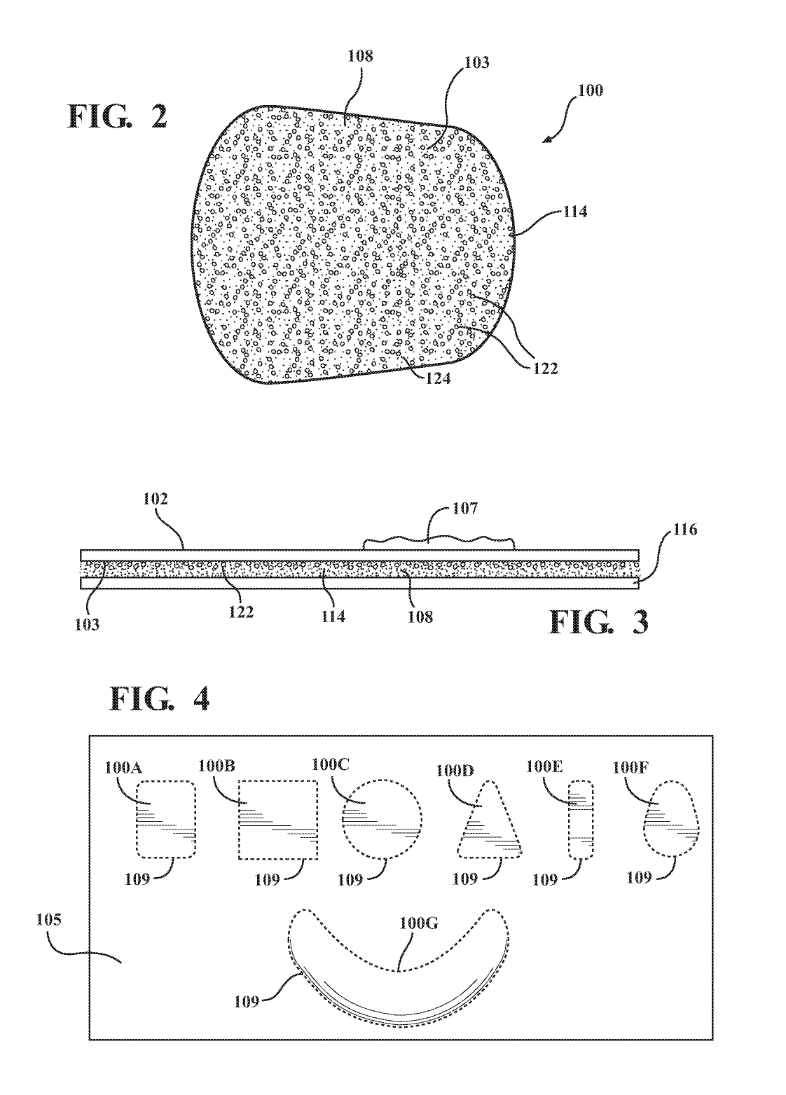

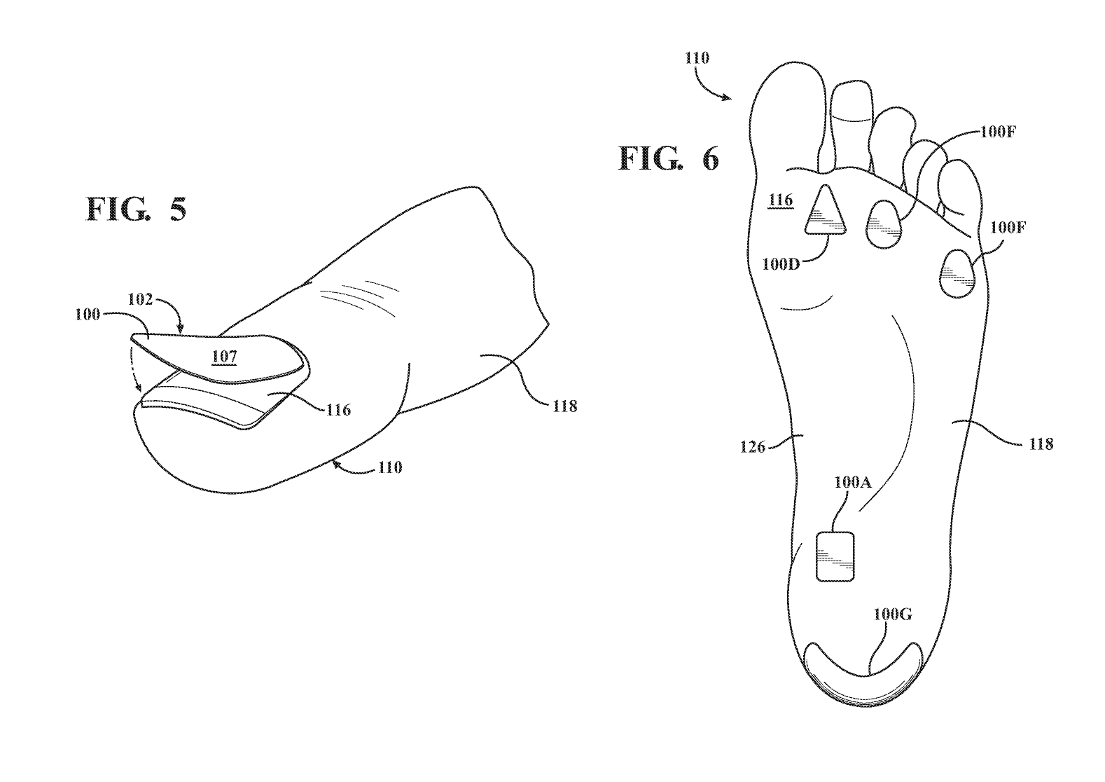

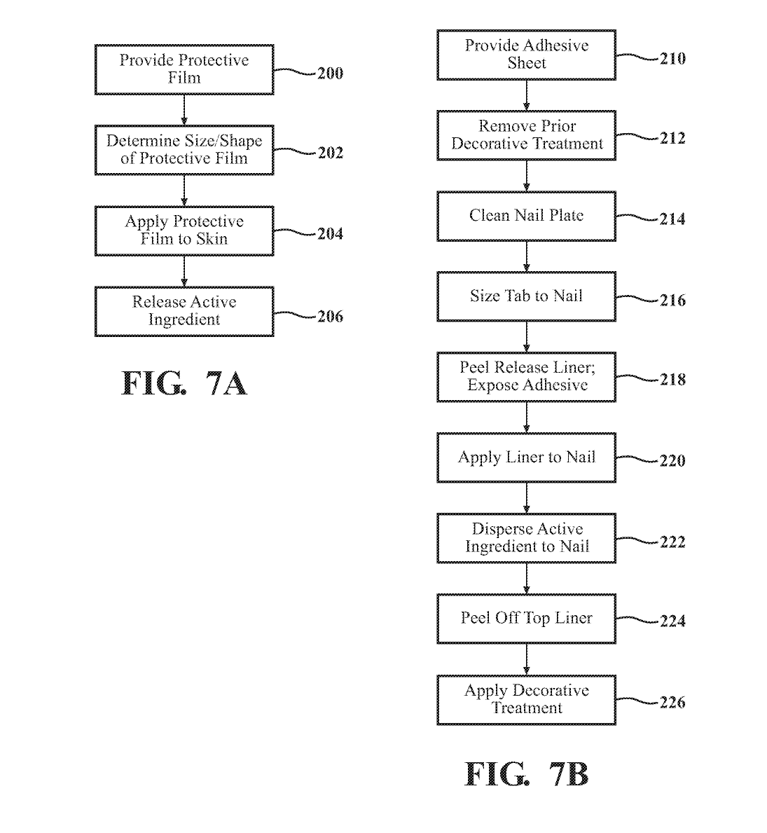

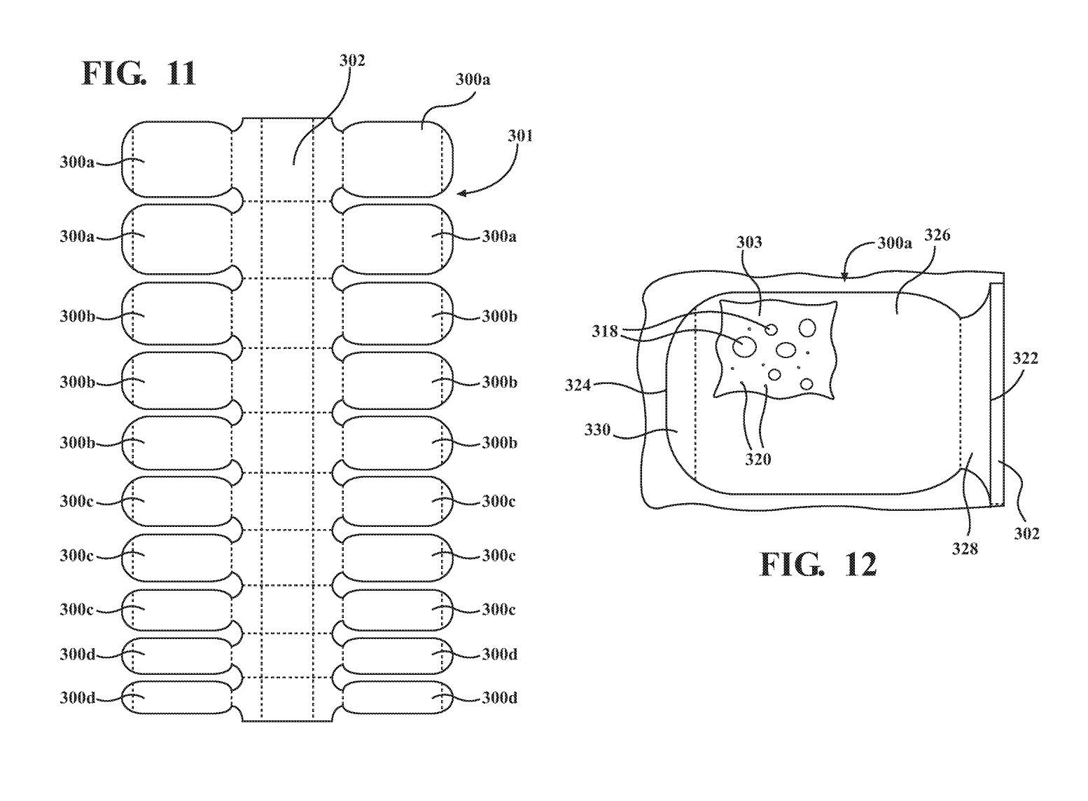

With reference to the Figures, FIGS. 2-4 illustrate the protective film 100 for application on the individual's body in its broadest aspects, FIGS. 5-6 illustrate examples of application of the protective film to different parts of the individual's body, FIG. 7 is a flowchart of the method for applying the protective film 100 described in FIGS. 2-4, FIGS. 8-10 illustrate a carrying sheet 130 with a protective film 100 for application on the individual's body and in particular, the individual's nail 116 and FIG. 10 illustrates a protective film 200 for application to a user's heel. FIG. 11 is a schematic illustration of a carrying sheet with a plurality of sets of protective films for application to an individual's nails.

With reference to FIGS. 2-3, the protective film 100 can include a first side 102 and a second side 103. The first side 102 may have a smooth surface for application of a decorative treatment 107 thereto. It will be appreciated that the first side 102 may also have a texture or other surface characteristic that facilitates or allows receipt of a decorative treatment 107. According to an aspect, the decorative treatment 107 may consist of a lacquer nail polish, a gel nail polish, a second adhesive and an artificial nail, or an acrylic overlay with or without a nail tip. It will be appreciated that a variety of other decorative treatments 107 may also be employed. According to an aspect, the second side 103 may have an adhesive 108 thereon for securing the protective film 100 to the individual's body. According to an aspect, the adhesive 108 can include an active ingredient 114 interspersed therein. The active ingredient 114 may be one or more health additives 114 that are arranged to be dispersed onto and into the portion of the individual's body 110 where the protective film 100 is applied. The active ingredient 114 may be mixed in with or otherwise dispersed adjacent to the adhesive 108. The portion of the individual's body 110, in which the protective film 100 is applied, may be the individual's nail 116 (fingernail or toe nail, schematically shown in FIG. 3). According to another aspect, the film 100 may be applied to a portion of the individual's skin 118 such as on a callus, a blister, or a crack formed therein.

According to one aspect, the active ingredient 114 may be time-released such that the active ingredient 114 is dispersed onto the portion of the individual's body 110 after or for a predetermined amount of time following application of the protective film 100 to the individual's body. In another aspect, the active ingredient 114 may be time-released such that the active ingredient 114 is dispersed continuously onto the portion of the individual's body 110 immediately when applied or after a predetermined amount of time following application, until the protective film 100 is removed. The active ingredients 114 may include one or more health additives 114 including vitamins, minerals, proteins, nutrients, topical medications, homeopathic treatments, repairing additives, or a combination thereof for enhancing, improving, or repairing the condition of the individual's nail 116 or skin 118. Such health additives 114 may include, but are not limited to, salicylic acid, urea, or carbamide. For example, where the protective film 100 is applied to a callus, the active ingredient 114 may include a first health additive 114 such as salicylic acid and any number of other health additives. Additionally, in a further example where the protective film 100 is applied to a callus, the adhesive 108 may be infused with an active ingredient 114 that is adapted to assist in isolating the callus, such that callus becomes partially or entirely attached to the protective film 100, and detached from the individual's skin 118 when the protective film 100 removed. Accordingly, the individual may see part of, or their entire callus stuck to the used protective film 100 when it is removed.

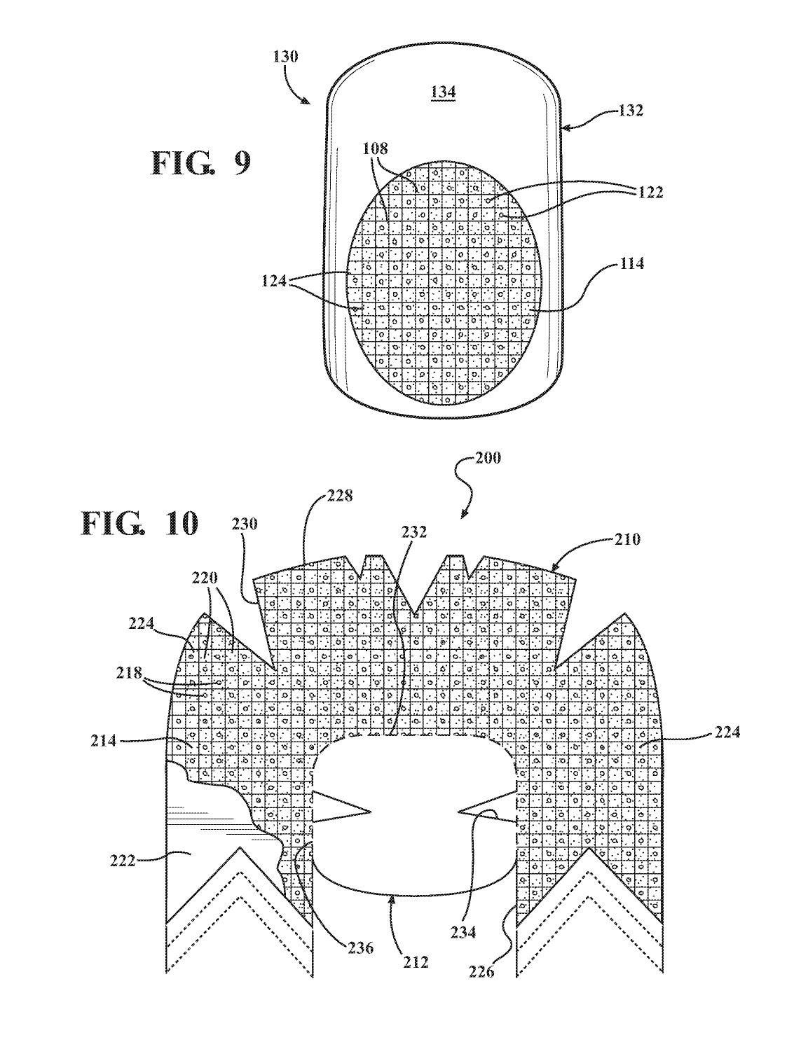

According to still another aspect best presented in FIG. 9, the adhesive 108 may be arranged on the film 100 as a plurality of beads 122 spaced apart from one another by a plurality of gaps 124 such that the plurality of beads 122 and the plurality of gaps 124 may form a grid-like pattern. It will be appreciated that the adhesive 108 may be arranged on the film 110 in a variety of different ways and patterns. According to an aspect, the plurality of beads 122 may entrap time-released active ingredient 114. In an alternative aspect, the plurality of adhesive beads 122 may entrap non-time-released active ingredients 114 such that the active ingredients are immediately dispersed onto the portion of the individual's body 110 upon application.

In one aspect, as best shown in FIG. 4, the protective film 100 may be defined as one or more pre-cut portions 100a-g formed in pre-determined shapes to accurately fit one or more particular portions of the individual's body 110. In order to provide the pre-cut portions the protective film 100 may be formed as a sheet 105 with the pre-cut portions 100a-g removeably arranged thereon. In order to removeably attached the protective film portions 100a-g to the film sheet 105, each protective film portion 100a-g on the film sheet 105 may have one or more perforated edges 109 to indicate the pre-cut or pre-determined shapes such that the pre-cut portions 100a-g may easily and individually be removed from the film sheet 105. For instance, the protective film portion 100a-g may have a nail-like shape for application to the individual's nail. In other examples, the protective film portion 100a-g may have a rectangular (100a), square (100b), circular (100c), triangular (100d), oval (100e), oblong (100f), or U-like (100g) shape for application to the individual's skin. It will be appreciated that the film portions 100a-g may be configured in a variety of other suitable shapes. FIG. 6, illustrates the U-liked shaped protective film portion 100g applied to an individual's heel. Additionally, the triangular shaped film portion 100d is applied to the bottom of the individual's foot, where a callus may be located. It should be appreciated that the protective film 100 may be selectively configured to have the same shape as the callus on the individual's skin or to have the same shape as other specific areas of the body 110.

FIG. 5 shows the protective film 100, as discussed above and as shown in FIGS. 2-4, in the process of being applied to an individual's nail 116. The method or process for application of the protective film 100 to the individual's body 110 and in particular, to the individual's nail 116 as shown in FIG. 5 and the individual's foot 126 as shown in FIG. 6, is described in further detail below, as depicted in FIG. 7. It will be appreciated that the described method is merely exemplary and that a variety of other suitable methods may be employed.

With reference to FIG. 7, the method may include 200 providing the protective film 100 for application to the individual's body 110, 202 instructing the individual to apply the protective film 100 to the portion of an individual's body 110, and 204 dispersing one or more active ingredients 114 onto the portion of the individual's body 110 where the protective film 100 is applied. As discussed above, the active ingredients 114 may be a health additive that is configured to improve the condition of the portion of the individual's body 110 where the protective film 100 is applied as discussed above. In application, the protective film 100 may provide a barrier from damage associated with the application of a decorative treatment 107, the removal process of the decorative treatment, or one or more external elements. As shown in FIG. 7A, step 200 of providing the protective film 100 may further include 201 determining a specific shape of a protective film portion 100a-g based on the portion of the individual's body 110 in which the film portion 100a-g is being applied. The shape of the film portion 100a-g may be predetermined or pre-cut. Alternatively, the sheet 105 may be configured such that an individual could cut the film 100 to a predetermined shape. As shown in FIG. 7B, the method may further include 203 removing a backing membrane 111 from the second side 103 of the protective film 100. The backing membrane may overlie and protect the adhesive 108 and active ingredient 114 from exposure prior to application to the portion of the individual's body 110.

As illustrated in FIG. 5, the system and method can be utilized in connection with an individual's finger or toe nails 116. According to this aspect, the system can include applying a thin film 100 over an individual's nail 116. The film 100 can be sized so that a single sized film 100 can be used on each nail 116. According to an aspect, the film 100 may be applied from the individual's cuticle to the tip of the nail 116, as also shown in FIG. 5, or vice versa. According to still another aspect, films 100 in different sizes (i.e., small, medium, or large) can also be employed. The film 100 may be a topical film and may be constructed of a membrane material, as shown in FIGS. 2-4. It will be appreciated that the film 100 may be constructed of a variety of other suitable materials.

It is a further aspect of the present disclosure that a protective film 100 as applied to the individual's skin 118 is the same protective film or may have similar characteristic to the protective film 100 as applied to the individual's nail 116, described above. The protective film 100 and method also can be utilized in connection with the individual's skin 118 on any portion of the individual's body 110. In particular, the protective film 100 may be applied to the individual's skin 118 prior to or after any damage, such as cracks, blisters, or calluses, has occurred. The protective film 100 may be designed to act as a protective shield on the skin 118 and may also act as a repairing agent after damage has occurred. According to an aspect, the system can include applying a thin film 100 over a portion of the individual's skin 118 on the individual's body 110. The film 100 can be sized so that a single sized film can be used on the portion of the individual's skin 118. The film 100 may have various sizes (small, medium, and large) and may further include multiple perforations 109 that allow the film 100 to be sized easily for different portions of the individual's body 110, as shown in FIG. 6. The film 100 may be a topical film and may be constructed of membrane material. It will be appreciated that the film 100 may alternatively be constructed of a variety of other suitable materials.

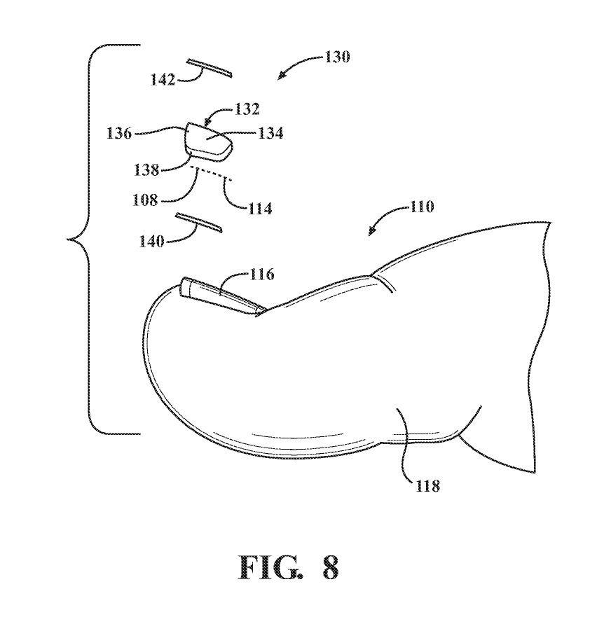

With reference to FIG. 8, an artificial nail or artificial nail tip 132 may be provided. The artificial nail tip 132 may have a body 134 with an upper surface 136 and a lower surface 138. The nail body 134 may be formed of an acrylic material, however, other materials could be utilized. an adhesive is 108 disposed against the lower surface 138 for securing the nail body 134 to a nail 116 of an individual. As discussed with the films 100 above, the adhesive 108 may be made of a plurality of beads 122 spaced apart from one another by a plurality of gaps 124. The plurality of beads 122 may include an active ingredient 114 such as one or more health additives 114 for dispersion onto the individual's nail 116. According to an aspect, a protective film 100 may also be provided to form a barrier between the artificial nail 132 and the individual's nail 116 to protect the individual's nail 116 from damage caused by the application of decorative treatments 107 on the artificial nail 132 or the removal of the artificial nail 132 from the individual's nail 116. When disposed on a fingernail 116, the apparatus 130 not only provides for an aesthetically pleasing covering of the fingernail 116, but also protects the fingernail 116 and delivers at least one active ingredient 114 to the fingernail 116. As illustrated, the lower surface 138 of the nail tip 132 may be sized and configured to overlie and be secured to the fingernail 116. In one example, a portion of the lower surface 138 engages the natural fingernail 116, and another portion extends over and away from the fingernail 116. It should be appreciated that the nail body 134 could have other shapes and sizes, e.g., the boundaries of the nail body 134 may be shaped and sized such that they are flush with the perimeter of the tip of the natural fingernail 116. The upper surface 136 may receive one or more coatings such as nail polish 142 and finishing materials. The nail polish 142 can be used to make the apparatus 130 look like the natural fingernail 116 or it can also be used to provide other attractive and unique appearances. The finishing material can be used to protect the natural fingernail 116 and/or the nail polish 142 from damage. According to an aspect, the upper surface 136 may be coated with the nail polish 142 while the nail tip 132 is packaged.

A backing membrane 140 may removeably overly the adhesive 108 and the active ingredient 114 on the lower surface 138 for covering and protecting the adhesive 108 and the active ingredient 114 prior to removal of the backing membrane 140 and subsequent placement of the lower surface 138 of the nail body 134 on the natural fingernail 116. Said another way, in an initial configuration, the backing membrane 140 overlies and protects the adhesive 108 and active ingredient 114. The backing membrane 140 may be removed from the nail body 134 into an exposed configuration in which the adhesive 108 and active ingredient 114 are exposed to allow the adhesive 108 and active ingredient 114 to be placed over the natural fingernail 116. The backing membrane 140 also ensures that that active ingredient 114 and adhesive 108 are preserved prior to placement of the apparatus 130 onto the natural fingernail 116.

According to a further aspect of the disclosure presented in FIG. 10, an embodiment of the protective film 200 may include a heel patch portion 210 that is generally U-shaped such that it is adapted for surrounding the heel of a user to remove calluses therefrom. According to an aspect, the protective film 200 may also include a toe and ball portion 212 for being positioned over the toes or ball of the user's foot to remove calluses therefrom. The protective film 200 also includes a top surface 214 and a bottom surface opposite the top surface 214. The top surface 214 includes an adhesive 218 for gripping the user's foot. An active ingredient 220 such as a health additive, repairing additive, vitamin, or medication is mixed with the adhesive 218 for being deposited onto a callus on the user's foot to treat the callus. A backing membrane 222 may overly the adhesive 218 and active ingredient 220 prior to placement on the user's foot. The U-shape of the heel patch portion 210 generally is defined by a pair of parallel legs 224 and a pocket 226 therebetween. The heel patch portion 210 has an outer perimeter 228 which defines a plurality of irregularities 230 to allow the heel patch portion 210 to conform to the shape of the user's heel. The toe and ball portion 212 may be positioned central to the heel patch portion 210 in the pocket 226 and is connected to the heel patch portion 210 via a perforated edge 232 such that it may easily be disconnected from the heel patch portion 210 prior to use. It should be appreciated that this arrangement provides compact and simple packaging of the protective film 200. The toe and ball portion 212 may also include a plurality of irregularities 234 defined on a perimeter 236 thereof to allow the toe and ball portion 212 to conform to a user's toes or ball of their foot.

According to a further aspect of the disclosure, the active ingredients 220 of the protective patch 200 are configured to eliminate calluses from the individual's foot. In the example embodiment, the active ingredient 220 may include a solution of water and glycerin with the following ingredients contained therein: mandelic acid at 10-20% in weight, salicylic acid at 2% in weight and bromelain at 10-25% in weight. [or potassium hydroxide and other additives (same as the summary); not sure I fully grasp this]

With reference to FIG. 11, a nail runner 301 is provided which includes a plurality of fingernail shaped protective film portions 300a-d. More particularly, the nail runner 300 includes an elongated central hub portion 302 and the plurality of nail-shaped protective film portions 300a-d which are connected to the hub portion 302. As shown, the protective film portions 300a-d can be of various sizes and shapes such that they are adapted to be positioned on different finger nails. For example, as shown in FIG. 11, four of the protective film portions 300a-d may be thumb portions 300a that are sized and shaped for being positioned over a thumb nails, six of the protective film portions 300a-d are middle portions 300b that are sized and shaped for being positioned over middle finger nails, six of the protective film portions 300a-d are index/ring portions 300c which are sized and shaped for being positioned over index and ring finger nails, and four of the protective film portions 300a-d are pinky portions 300d which are sized and shaped for being positioned over pinky finger nails. It should be appreciated that more or fewer of any of the protective film portions 300a-d may be utilized, e.g., additional index/ring portions 300c may be provided because they are used for more heavily than the rest. It should be appreciated that due to the arrangement of the runner 301, smaller protective film portions 300a-d may be utilized as compared to runners of the prior art.

As best illustrated in FIG. 12, each of the protective film portions 300a-d includes a top side (opposite the page) and a bottom side 303. As discussed above, the top side may include a smooth surface for application of a decorative treatment thereto, or may include textures or other surface characteristic that facilitate or allow receipt of a decorative treatment. The bottom side 303 may have an adhesive 318 thereon for securing the protective film portion 300a-d to the user's nails. Furthermore, the bottom side 303 may have an active ingredient 320 interspersed with the adhesive 318. As previously discussed, the active ingredient 320 may be one or more health additives that are arranged to be dispersed onto and into the nail. According to an aspect of the disclosure, the adhesive 318 and active ingredient 320 may be provided in the form of a hydrofilm. Each protective film 300a-d portion extends between a first edge 322 that is attached to the hub portion 302 and a second edge 324 opposite the hub portion 302. A backing membrane 326 may overly the adhesive 318 and active ingredient 320 prior to placement on the user's foot. As illustrated, the backing membrane 326 of each of the protective film portions 300a-d includes a first handle 328 at the first edge 322, adjacent to the hub portion 302, and a second handle 330 at the second edge 324. The first and second handles 328, 330 may each be configured as a flap 328, 330 that extends upwardly from the backing membrane 326 which may be gripped by a user to allow the removal of the backing membrane 326 from the protective film portion 300a-d. Due to the strong adhesive characteristics of hydrofilm, a proper application of force needs to be applied directly to the nail protective film portion 300a-d and pressed into place against the nail without the adhesive being touched. The arrangement of the backing membrane 326 with two handles 328, 330 advantageously allows easy removal of the backing membrane 326 from protective film portion 300a-d without the nail being touched through the application process. For example, the second handle 330 may be gripped and the backing membrane 326 may be removed in the direction of the cuticle of the nail as the protective film portion 300a-d is placed on the nail, or the first handle 330 may be gripped and the backing membrane 326 may be remove in the direction of the tip of the nail as the protective film portion 300a-d is placed on the nail.

Note that not all of the activities described above in the general description or the examples are required, that a portion of a specific activity may not be required, and that one or more further activities may be performed in addition to those described. Still further, the orders in which activities are listed are not necessarily the order in which they are performed.

The specification and illustrations of the embodiments described herein are intended to provide a general understanding of the structure of the various embodiments. The specification and illustrations are not intended to serve as an exhaustive and comprehensive description of all of the elements and features of apparatus and systems that use the structures or methods described herein. Many other embodiments may be apparent to those of skill in the art upon reviewing the disclosure. Other embodiments may be used and derived from the disclosure, such that a structural substitution, logical substitution, or another change may be made without departing from the scope of the disclosure. Accordingly, the disclosure is to be regarded as illustrative rather than restrictive.

Certain features are, for clarity, described herein in the context of separate embodiments, may also be provided in combination in a single embodiment. Conversely, various features that are, for brevity, described in the context of a single embodiment, may also be provided separately or in any sub combination. Further, reference to values stated in ranges includes each and every value within that range.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

The above-disclosed subject matter is to be considered illustrative, and not restrictive, and the appended claims are intended to cover any and all such modifications, enhancements, and other embodiments that fall within the scope of the present invention. Thus, to the maximum extent allowed by law, the scope of the present invention is to be determined by the broadest permissible interpretation of the following claims and their equivalents, and shall not be restricted or limited by the foregoing detailed description.

Although only a few exemplary embodiments have been described in detail above, those skilled in the art will readily appreciate that many modifications are possible in the exemplary embodiments without materially departing from the novel teachings and advantages of the embodiments of the present disclosure. Accordingly, all such modifications are intended to be included within the scope of the embodiments of the present disclosure as defined in the following claims. In the claims, means-plus-function clauses are intended to cover the structures described herein as performing the recited function and not only structural equivalents, but also equivalent structures.

What is claimed is:

1. A protective film for application to a portion of an individual's body, comprising:
a heel patch portion including a first surface and a second surface opposite the first surface;
an adhesive disposed on the first surface for securing the protective film to the portion of the individual's body;
an active ingredient disposed on the first surface and mixed with the adhesive and configured to be disposed onto the portion of the individual's body where the protective film is applied; and
wherein the heel patch portion has a U-shape for surrounding a heel of the individual.

2. The protective film of claim 1, wherein the heel patch portion has an outer perimeter defining a plurality of irregularities for allowing the callus remover patch portion to conform to the shape of a user's heel.

3. The protective film of claim 1, wherein the heel patch portion includes a pair of legs extending generally parallel with one another and defining a pocket therebetween; and wherein a toe and ball portion is positioned central to the heel patch portion in the pocket.

4. The protective film of claim 3, wherein the toe and ball portion is connected to the heel patch portion via a perforated edge such that it may easily be disconnected from the heel patch portion prior to use.

5. The protective film of claim 4, wherein the toe and ball portion has a perimeter presenting a plurality of irregularities to allow the tow and ball portion to conform to an individual's toes or ball of their foot.

6. The protective film of claim 4, wherein a backing membrane removeably overlies the adhesive and health additive.

7. The protective film of claim 1 wherein the active ingredient includes a solution of water and glycerin with the following ingredients contained therein: mandelic acid at 10-20% in weight, salicylic acid at 2% in weight and bromelain at 10-25% in weight.

8. A protective film for application to a portion of an individual's body, comprising:
a first surface and a second surface opposite the first surface; and
an active ingredient disposed on the first surface and configured to be disposed onto the portion of the individual's body where the protective film is applied;
wherein the active ingredient includes a solution of water and glycerin with the following ingredients contained therein: mandelic acid at 10-20% in weight, salicylic acid at 2% in weight and bromelain at 10-25% in weight.

9. A protective device for application to a portion of an individual's body, comprising:
a runner including an elongated central hub portion and a plurality of protective film portions extending from the central hub;
each of the protective film portions including a first edge at the central hub portion and a second edge opposite the hub portion;
each of the protective film portions including a first side and a second side opposite the first side;
an active ingredient disposed on the second side of each of the protective film portions and configured to be disposed onto the portion of the individual's body where the protective film is applied;
an adhesive disposed on the second side of protective film portions for securing the protective film portion to the individual's body;
a backing membrane overlying the adhesive and the active ingredient of each of the protective film portions; and
each of the protective film portions including a first handle at the first edge and a second handle at the second edge for allowing the individual to remove the backing membrane from the second side of the protective film portion.

10. The protective device as set forth in claim 9 wherein each of the protective film portions is shaped liked a finger nail.

11. The protective device as set forth in claim 10 wherein the plurality of protective film portions includes a plurality of different sizes.

12. The protective device as set forth in claim 10 wherein the first and second handles are each a flap extending from the backing membrane.
